(19) United States
(12) Patent Application Publication
(10) Pub. No.: US 2017/0300186 A1
(43) Pub. Date: Oct. 19, 2017

(54) SYSTEMS AND METHODS FOR HEALTH MANAGEMENT

(71) Applicants: Peter Kuhar, Palo Alto, CA (US); Tom Xu, El Cerrito, CA (US); Eric Huynh, Alhambra, CA (US); Tina Tavcar, Ljubljana (SI); Bojan Bostjancic, Palo Alto, CA (US)

(72) Inventors: Peter Kuhar, Palo Alto, CA (US); Tom Xu, El Cerrito, CA (US); Eric Huynh, Alhambra, CA (US); Tina Tavcar, Ljubljana (SI); Bojan Bostjancic, Palo Alto, CA (US)

(21) Appl. No.: 15/131,321

(22) Filed: Apr. 18, 2016

(51) Int. Cl. G06F 3/0482; G06F 3/0484; G06F 3/0481; G06T 11/20; A61B 5/145

(52) U.S. Cl. CPC G06F 3/0482; G06T 11/206; A61B 5/14532; G06F 3/04817; G06F 3/04842

(57) ABSTRACT

Described herein are systems and methods of diabetes management and prediabetes prevention. The method includes rendering a graphical user interface including a first plurality of hexagonal tiles arranged in a honeycomb pattern, the first plurality of hexagonal tiles together displaying tracked data for a plurality of datatypes; receiving a tracked data input for a first datatype; adding the input data to a new or an existing hexagonal tile representing a tracked data point of the first datatype; dynamically rearranging the first'plurality of hexagonal tiles to maintain a time-ordered sequence of tracked data; receiving a user selection of a tile of a second datatype; and displaying a plurality of icons representing selectable user options corresponding to the user-selected tile. In some embodiments, the system tracks a glucose level, an administered medication value, a pedometer step count, and a food consumption input.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This invention relates generally to the consumer electronics and health and lifestyle monitoring fields, and more specifically to new and useful systems and methods for health tracking.

### BACKGROUND

Health monitoring and management systems are commonplace in today's consumer market. For example, applications exist for tracking heart rate, weight, food consumption, workouts, health conditions (e.g., diabetes, psoriasis, obesity, etc.), and a variety of other parameters and/or conditions. However, to track and manage a global health picture, a user must use multiple applications, each with unique use cases, user inputs, and navigational tools, adding unnecessary complexity to the process. It can be difficult to share the tracked data with health care providers and coaches. Further, current applications fail to provide a direct, complete, and dynamic view of a user's global health in which a user can dictate which aspects of his/her health are tracked and shared.

Thus, there is a need for new and useful systems and methods for health tracking. This invention provides such new and useful systems and methods.

### SUMMARY

The present disclosure is directed to health monitoring and management systems. One aspect of the disclosure is directed to a diabetes management and prediabetes prevention system. In some embodiments, a diabetes management and prediabetes prevention system comprises a display; a processor communicatively coupled to the display; and a computer-readable medium having non-transitory, processor-executable instructions stored thereon, wherein execution of the instructions causes the processor to perform a method. In some embodiments, the method includes rendering a graphical user interface (GUI) comprising a first plurality of hexagonal tiles arranged in a honeycomb pattern, each hexagonal tile representing a tracked data point, the first plurality of hexagonal tiles together displaying tracked data for a plurality of datatypes; receiving a tracked data input for a first datatype, wherein the system is configured to receive tracked data inputs for at least the following datatypes: a glucose level, an administered medication value, a pedometer step count, and a food consumption input; adding the input data to a new or an existing hexagonal tile representing a tracked data point of the first datatype; dynamically rearranging the first plurality of hexagonal tiles to maintain a time-ordered sequence of tracked data; receiving a user selection of a tile of a second datatype; displaying a plurality of icons representing selectable user options corresponding to the user-selected tile, including an icon for a filtered view and an icon for a trends view; and displaying the view corresponding to the icon selected by the user.

In some embodiments, the filtered view comprises the honeycomb pattern with some of the first plurality of hexagonal tiles removed and rearranged so as to only display the user-selected tile and other hexagonal tiles representing tracked data of the second datatype. In some embodiments, the trends view comprises a graph of tracked data of the second datatype plotted against time or against tracked data of a different datatype specified by the user.

In some embodiments, the input is a user input. In some embodiments, the input is data imported from a wirelessly connected device. In some embodiments, the input is data acquired from a component of the mobile computing device.

In some embodiments, the display, processor, and computer-readable medium comprise components of a mobile computing device. In some embodiments, the mobile computing device comprises a laptop, mobile phone, or tablet.

In some embodiments, some or all of the tracked data is shareable with a diabetes management adviser or prediabetes prevention adviser.

In some embodiments, the system is configured to transmit and display messages sent between the diabetes management adviser or prediabetes prevention adviser and the user.

In some embodiments, the system further comprises additional non-transitory, processor-executable instructions stored on the computer-readable medium, wherein execution of the additional instructions causes the processor to perform a method. In some embodiments, the method includes displaying a second plurality of hexagonal tiles arranged in the honeycomb structure of the GUI, the second plurality of hexagonal tiles representing recommended trackable datatypes. In some embodiments, each of the second plurality of hexagonal tiles is selectable to input and display data of the datatype represented by the selected tile.

In some embodiments, the second plurality of hexagonal tiles represent trackable datatypes recommended for the user by a diabetes management adviser or prediabetes prevention adviser. In some embodiments, the first datatype is the second datatype. In some embodiments, the first and second datatypes are different datatypes.

In some embodiments, the system further comprises a blood glucose monitor communicatively coupled to the processor.

One aspect of the invention is directed to a method of diabetes management and prediabetes prevention. In some embodiments, the method includes rendering a GUI comprising a first plurality of hexagonal tiles arranged in a honeycomb pattern, each hexagonal tile representing a tracked data point, the first plurality of hexagonal tiles together displaying tracked data for a plurality of datatypes; receiving a tracked data input for a first datatype, wherein the system is configured to receive tracked data inputs for at least the following datatypes: a glucose level, an administered medication value, a pedometer step count, and a food consumption input; adding the input data to a new or an existing hexagonal tile representing a tracked data point of the first datatype; dynamically rearranging the first plurality of hexagonal tiles to maintain a time-ordered sequence of tracked data; receiving a user selection of a tile of a second datatype; displaying a plurality of icons representing selectable user options corresponding to the user-selected tile, including an icon for a filtered view and an icon for a trends view; and displaying the view corresponding to the icon selected by the user.

In some embodiments, the method further includes transmitting one or more tile updates to a diabetes management adviser or prediabetes prevention adviser.

In some embodiments, the method further includes transmitting and displaying messages sent between the diabetes management adviser or prediabetes prevention adviser and the user.

In some embodiments, the method further includes displaying a second plurality of hexagonal tiles arranged in the honeycomb structure of the GUI, the second plurality of hexagonal tiles representing recommended trackable datatypes. In some embodiments, each of the second plurality of hexagonal tiles is selectable to input and display data of the datatype represented by the selected tile. In some embodiments, the second plurality of hexagonal tiles represent trackable datatypes recommended for the user by a diabetes management adviser or prediabetes prevention adviser.

In some embodiments, the method further includes receiving the tracked data input from a glucose monitor or insulin delivery device.

In some embodiments, the method further includes receiving a recommendation for a trackable datatype from a diabetes management adviser or prediabetes prevention adviser.

Another aspect of the invention is directed to a mobile health tracking system. In some embodiments, a mobile health tracking system comprises a display; a processor communicatively coupled to the display; and a computer-readable medium having non-transitory, processor-executable instructions stored thereon, wherein execution of the instructions causes the processor to perform a method. In some embodiments, the method includes rendering a GUI comprising a plurality of hexagonal tiles arranged in a honeycomb pattern, each hexagonal tile representing a tracked data point or dataset, the plurality of hexagonal tiles together displaying tracked data for a plurality of datatypes; receiving an input of tracked data of a first datatype; adding the input data to an existing hexagonal tile representing a tracked dataset of the first datatype or adding the input data to a new hexagonal tile representing a tracked data point of the first datatype; dynamically rearranging the plurality of hexagonal tiles to maintain a time-ordered sequence of tracked data; receiving a user selection of a tile of a second datatype; displaying a plurality of icons representing selectable user options corresponding to the user-selected tile, including an icon for a filtered view and an icon for a trends view; and displaying the view corresponding to the icon selected by the user.

One aspect of the invention is directed to a heart health mobile tracking system. In some embodiments, a heart health mobile tracking system comprises a display; a processor communicatively coupled to the display; and a computer-readable medium having non-transitory, processor-executable instructions stored thereon, wherein execution of the instructions causes the processor to perform a method. In some embodiments, the method includes rendering a GUI comprising a plurality of hexagonal tiles arranged in a honeycomb pattern, each hexagonal tile representing a tracked data point or dataset, the plurality of hexagonal tiles together displaying tracked data for a plurality of datatypes; receiving a tracked data input comprising a signal of a user's heart beat over time, wherein the signal is acquired while the use is in a sitting position and a standing position; calculating a plurality of heart health metrics comprising a heart rate, a heart rate variability, and a standup test result from the tracked data input; adding each of the heart health metrics to a separate new hexagonal tile within the honeycomb pattern; dynamically rearranging the plurality of hexagonal tiles to maintain a time-ordered sequence of tracked data; receiving a user selection of a tile of a desired datatype; displaying a plurality of icons representing selectable user options corresponding to the user-selected tile, including an icon for a filtered view and an icon for a trends view; and displaying the view corresponding to the icon selected by the user.

One aspect of the invention is directed to a mobile weight management system. In some embodiments, a mobile weight management system comprises a display; a processor communicatively coupled to the display; and a computer-readable medium having non-transitory, processor-executable instructions stored thereon, wherein execution of the instructions causes the processor to perform a method. In some embodiments, the method includes rendering a GUI comprising a plurality of hexagonal tiles arranged in a honeycomb pattern, each hexagonal tile representing a tracked data point or dataset, the plurality of hexagonal tiles together displaying tracked data for a plurality of datatypes; receiving an input of a user's current weight; receiving a user input of a target weight; calculating a daily caloric budget for the user; automatically detecting a user step count; adding the detected user step count to an existing hexagonal tile representing daily cumulative user step count; determining a count of calories burned from the detected user step count; receiving a user input indicative of food or beverage consumption; creating a new hexagonal tile representing the food or beverage consumption; determining a calorie count of the food or beverage consumption; dynamically rearranging the plurality of hexagonal tiles to maintain a time-ordered sequence of tracked data; and updating a remaining daily caloric budget for the user.

In some embodiments, the system further includes additional non-transitory, processor-executable instructions stored on the computer-readable medium, wherein execution of the additional instructions causes the processor to perform a method. In some embodiments, the method includes receiving a user selection of a tile of a desired datatype; displaying a plurality of icons representing selectable user options corresponding to the user-selected tile, including an icon for a filtered view and an icon for a trends view; and displaying the view corresponding to the icon selected by the user.

One aspect of the invention is directed to a method of heart rate monitoring. In some embodiments, a method of heart rate monitoring includes rendering a GUT comprising a first plurality of tiles, each tile representing a tracked data point, the first plurality of tiles together displaying tracked data for a plurality of datatypes; acquiring, using a camera, a tracked data input for a first datatype, wherein the tracked data input comprises a video or sequence of images of a facial region or finger pad of a user; analyzing, using a processor, the video or sequence of images to determine one or more of a degree of coloration and a degree of transparency of the facial region or finger pad of the user; calculating, using the processor, a heart rate of the user based on the one or more of the determined degree of coloration and transparency; adding the input data to a new or an existing tile representing a tracked data point of the first datatype; dynamically rearranging the first plurality of tiles to maintain a time-ordered sequence of tracked data; receiving a user selection of a tile of a second datatype; displaying a plurality of icons representing selectable user options corresponding to the user-selected tile, including an icon for a filtered view and an icon for a trends view; and displaying the view corresponding to the icon selected by the user.

One aspect of the invention is directed to a method of sleep quality monitoring. In some embodiments, the method includes rendering a GUI comprising a first plurality of tiles, each tile representing a tracked data point, the first plurality of tiles together displaying tracked data for a plurality of datatypes; emitting, using one or more speakers, one or more inaudible sounds comprising a plurality of sound waves, wherein the plurality of sound waves interact with a user in proximity to the one or more speakers, and wherein the plurality of sound waves change in amplitude, time delay, and/or frequency after interaction with the user; acquiring, using one or more microphones, one or more sound signals comprising the changed plurality of sound waves; analyzing the one or more sound signals to determine an activity profile of the user; and adding the activity profile to a new or an existing hexagonal tile.

One aspect of the invention is directed to a GUI for presentation on a mobile computing device. In some embodiments, the GUI includes a first plurality of hexagonal tiles arranged in a honeycomb pattern, each hexagonal tile representing a tracked data point or dataset, the first plurality of hexagonal tiles together displaying tracked data for a plurality of datatypes. In some embodiments, the GUI is configured to dynamically rearrange the first plurality of hexagonal tiles in response to an input to maintain a time-ordered sequence of tracked data. In some embodiments, each of the first plurality of hexagonal tiles is configured to be activated by a user selection. In some embodiment, upon activation of a user selected tile of a first datatype, the GUI comprises a plurality of icons representing selectable user options, including an icon for a filtered view and an icon for a trends view, each icon configured to route a user to the corresponding view upon selection.

One aspect of the invention is directed to a method performed by a mobile computing device. In some embodiments, the method includes rendering a GUI comprising a first plurality of hexagonal tiles arranged in a honeycomb pattern, each hexagonal tile representing a tracked data point or dataset, the first plurality of hexagonal tiles together displaying tracked data for a plurality of datatypes; receiving an input of tracked data of a first datatype; adding the input data to an existing hexagonal tile representing a tracked dataset of the first datatype or adding the input data to a new hexagonal tile representing the tracked data point of a first datatype; dynamically rearranging the first plurality of hexagonal tiles to maintain a time-ordered sequence of tracked data; receiving a user selection of a tile of a second datatype; displaying a plurality of icons representing selectable user options corresponding to the user-selected tile, including an icon for a filtered view and an icon for a trends view; and displaying the view corresponding to the icon selected by the user.

### BRIEF DESCRIPTION OF THE DRAWINGS

FIG. 1A illustrates a schematic diagram of one embodiment of a system for mobile health tracking;

FIG. 1B illustrates a schematic diagram of one embodiment of a system for mobile health tracking;

FIG. 2 illustrates one embodiment of a graphical user interface for mobile health tracking;

FIG. 3 illustrates one embodiment of a graphical user interface for diabetes management and prediabetes prevention;

FIG. 4 illustrates one embodiment of a graphical user interface for heart health management;

FIG. 5 illustrates one embodiment of a graphical user interface for weight management;

FIG. 6 illustrates one embodiment of a graphical user interface depicting selectable user options;

FIG. 7 illustrates one embodiment of a graphical user interface depicting a filtered view;

FIG. 8A illustrates one embodiment of a graphical user interface depicting selectable user options for a trends view;

FIG. 8B illustrates one embodiment of a graphical user interface depicting a trends view;

FIG. 9 illustrates one embodiment of a graphical user interface depicting a new tile view;

FIG. 10 illustrates one embodiment of a graphical user interface depicting a competition view;

FIG. 11 illustrates a flow chart of one embodiment of a method of rendering and displaying a mobile health tracking graphical user interface;

FIG. 12 illustrates a flow chart of one embodiment of a method of diabetes management and prediabetes prevention using a mobile health tracking graphical user interface;

FIG. 13 illustrates a flow chart of one embodiment of a method of heart health management using a mobile health tracking graphical user interface; and

FIG. 14 illustrates allow chart of one embodiment of a method of weight management using a mobile health tracking graphical user interface.

FIG. 15 illustrates a flow chart of one embodiment of a method of heart rate monitoring; and

FIG. 16 illustrates a flow chart of one embodiment of a method of sleep quality monitoring.

### DETAILED DESCRIPTION

The following description of the preferred embodiments of the invention is not intended to limit the invention to these preferred embodiments, but rather to enable any person skilled in the art to make and use this invention. Disclosed herein are systems and methods for health tracking.

In general, the systems and methods described herein are intended for use by a user. As used herein, a user includes: any person who desires to track one or more personal health parameters; an athlete; a personal trainer; a diabetes patient; a person desiring to lose, maintain, or gain weight; a person suffering or recovering from a heart condition (e.g., coronary heart disease, heart attack, congestive heart failure, congenital heart disease, high cholesterol, hypertension, etc.); a person suffering from or managing a metabolic condition (e.g., diabetes); a prediabetic person (i.e., person with elevated levels of A1C or fasting glucose); a person desiring to track stress levels; a person training for an event (e.g., marathon, weight lifting, triathlon, biking, swimming, rowing, running, walking, etc.); a physician; a healthcare professional; a diabetes management adviser; a prediabetes prevention advisor; or any other individual desiring to track one or more datatypes, health-related parameters, and/or conditions.

In some embodiments, a user may create a profile in the system to indicate one or more attributes (e.g., height, weight, activity level, schedule, perceived stress level, etc.) of the user, a health condition of the user, one or more health priorities of the user (e.g., weight loss, toning, increasing water consumption, etc.), and/or one or more goals of the user. For example, a user may specify in his profile that he has diabetes, wants to achieve a target weight of 180 pounds, and needs to receive 40 units of insulin per day. Further, for example, a user may specify in her profile that she is training for a marathon and needs to reach an eight minute mile, drink ten glasses of water a day, and maintain a resting heart rate below 70 beats per minute.

In some embodiments of the systems and methods described herein datatypes are tracked. As used herein, tracking a datatype may include, but is not limited to, tracking steps in a time period (e.g., hour, day, week, month, etc.), amount of water consumed a number of calories consumed, a number of calories burned, an exercise route, a distance (e.g., miles, kilometers, feet, meters, etc.) traversed, an amount of weights lifted, number of repetitions completed, length of time of a workout or exercise, amount of time slept, a number of REM cycles achieved during sleep, number of snooze button pushes during a time period, rest period duration, exercise period duration, body fat percentage, current weight, desired weight, sleep efficiency, exercise moves, body measurements, a heart rate, a heart rate variability, a breathing rate, a pulse rate, a metabolic rate, a medication dose, a glucose level, or any other health parameter.

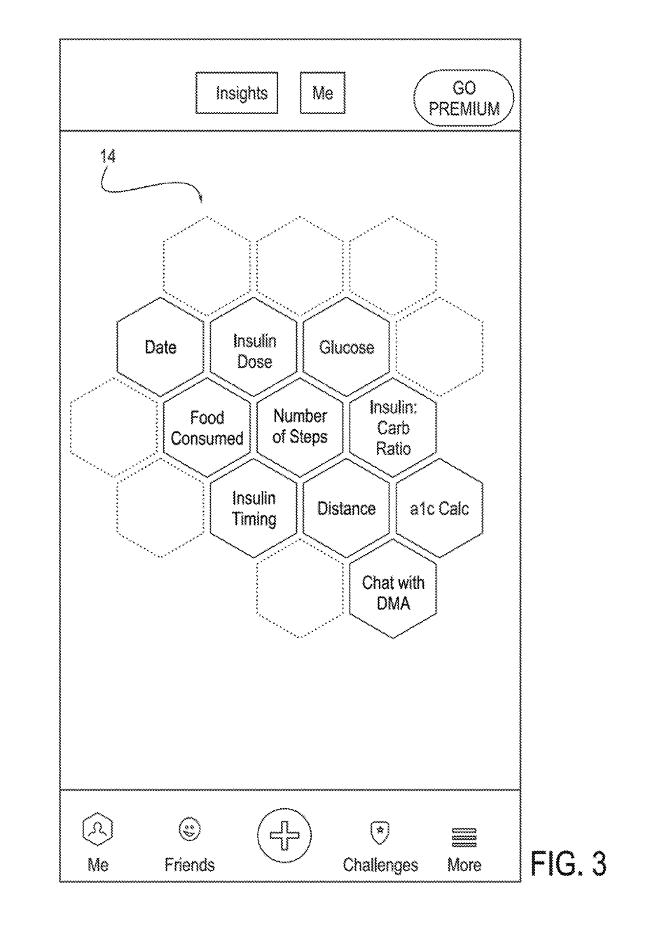

In some embodiments, tracking a datatype includes tracking one or more diabetes specific parameters, for example as shown in FIG. 3. In some embodiments, a diabetes specific parameter includes medication dose (e.g., insulin dose, glucagon dose, etc.), glucose level (e.g., blood, interstitial), a1c level, insulin-to-carbohydrate ratio, amount of food consumed, a timing of food consumption, amount of water consumed, number of steps in a time period, a distance of an exercise, number of hours of sleep, a number of REM cycles achieved during sleep, one or more hormone levels, or any other parameter.

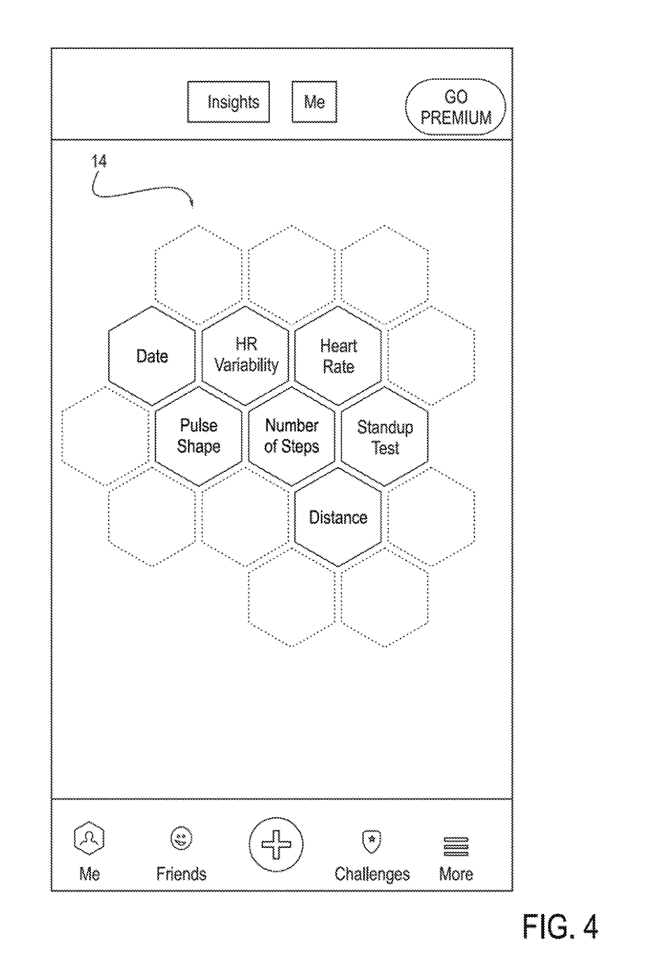

In some embodiments, tracking a datatype includes tracking one or more heart health parameters, for example as shown in FIG. 4. In some embodiments, a heart-health parameter includes heart rate, heart rate variability, pulse shape/waveform, stress level standup test result, cholesterol level, blood pressure, or any other heart health parameter.

In some embodiments, tracking a datatype includes tracking one or more weight management specific parameters, for example as shown in FIG. 4. In some embodiments, a weight management specific parameter includes tracking current weight, target weight, daily calorie budget, number of calories burned, amount of food consumed, number of calories consumed, number of steps in a time period, distance of an exercise, intensity of an exercise, number of repetitions completed, exercise schedule, completed exercises, or any other weight management parameter.

In some embodiments, tracking a datatype includes tracking one or more parameters related to a condition. For example, a condition may include a heart condition (e.g., coronary heart disease, heart attack, congestive heart failure, congenital heart disease, high cholesterol, hypertension, etc.), stressed state or anxiety, metabolic condition (e.g., diabetes, central pontine myelinolysis, hyperoxaluria, Trimethylaminuria, muscle metabolic diseases, hypothyroidism, etc.), weight-related condition (e.g., obesity, anorexia, bulimia, diabetes, hyperthyroidism, hypothyroidism, etc.), or any other condition. In some embodiments, one or more data points are collected for each datatype or health-related parameter tracked by the system. A data point may be an average of multiple points, a median of multiple points, an incremental counter, or one parameter measured at a discrete point in time. A data point may be a numerical value or image. Example data points include, but are not limited to, images of beverage(s) and/or food(s) consumed, distances, steps, routes, repetitions, target weight, current weight, calories consumed, calories burned, heart rate, sleep quality, pulse shape/waveform, task, and/or any other activity metric or value.

### System

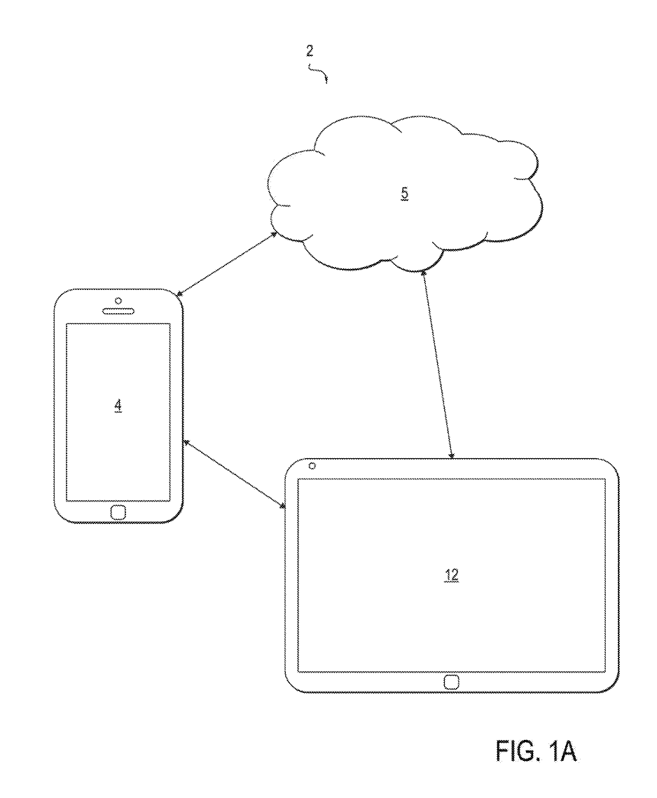

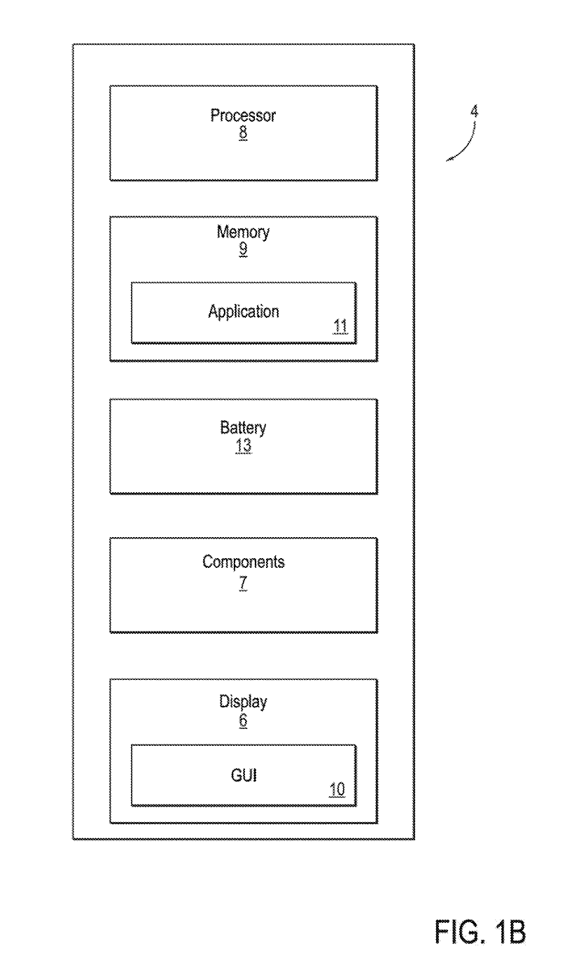

In some embodiments, as shown in FIG. 1A, a system 2 for mobile health tracking includes a computing device 4, a server 5, and an external device 12, for example for tracking a health related parameter. Various components of the system function to measure, transmit, and receive tracked health related parameters. In some embodiments, there is one-way or two-way communication between the computing device and the server, the computing device and the external device, and/or the server and the external device. The computing device, external device, and/or server may communicate wirelessly using Bluetooth, Wi-Fi, CDMA, LTE, other cellular protocol, other radiofrequency, or another wireless protocol.

In some embodiments, a computing device 4 is a stationary computing device. In some such embodiments, the stationary computing device includes a desktop computer. In some embodiments, as shown in FIG. 1A, a computing device 4 is a mobile computing device. In some such embodiments, the mobile computing device includes a mobile phone, tablet, laptop, netbook, notebook, or any other type of mobile computing device. In some embodiments, the computing device 4 is a computational device, wrapped in a chassis that includes a display (visual with or without touch responsive capabilities), a central processing unit (e.g., processor or microprocessor), internal storage (e.g., flash drive), n number of components (e.g., specialized chips and/or sensors), and n number of radios (e.g., WLAN, LTE, WiFi, Bluetooth, GPS, etc.).

In some embodiments, as shown in FIG. 1A, the system may include a server 5. The server may be a local server on the computing device or a remote server. In some embodiments, the server is a virtual server. In some embodiments, the server 5 may share data between the computing device 4 and the external device 12.

In some embodiments, the system 2 further includes an external device 2. The external device 12 may measure one or more datatypes and send, transmit, or export the one or more measurements to the computing device 4. The computing device may receive and/or import the data from the external device to analyze and/or display the data in one or more tiles. In some embodiments, sending or transmitting information occurs via a wired connection (e.g., IEEE 1394, Thunderbolt, Lightning, DVI, HDMI, Serial, Universal Serial Bus, Parallel, Ethernet, Coaxial, VGA, PS/2) or wirelessly (e.g., via Bluetooth, low energy Bluetooth, near-field communication, Infrared, WLAN, or other RF technology). The external device 12 may include a FitBit, a Pebble smartwatch, scale, a heart rate monitor (e.g., ECG, chest strap, etc.), pulse oximeter, continuous glucose monitor, interstitial glucose monitor, glucose pen, scanner e.g., for scanning barcodes of products for consumption), Apple Watch, a blood pressure cuff, caliper, pedometer, or any other device that can be used to measure one or more health parameters.

In some embodiments, as shown in FIG. 1B, a system for mobile health tracking includes a computing device 4 comprising a display 6 for presenting a graphical user interface (GUI) 10, a processor 8 communicatively coupled to the display 6, memory 9, and one or more components 7. The system functions to track and/or calculate one or more datatypes or health-related parameters. In some embodiments, the datatypes are tracked over time: in some embodiments, the datatypes are tracked one time, for example upon request by the user or upon detection of the occurrence of an event (e.g., walking, standing, sitting, injecting, eating, etc.).

The computing device 4 of some embodiments includes a processor 8, for example, a general purpose microprocessor. In some embodiments, the processor 8 is coupled, via one or more buses, to the memory 9 in order to read information from and write information to the memory 9. The memory 9 may be any suitable computer-readable medium that stores computer-readable instructions for execution by computer-executable components. In some embodiments, the computer-readable instructions include software stored in a non-transitory format, some such software having been downloaded as an application 11 onto the memory 9 of the mobile computing device 4. The processor 8, in conjunction with the software stored in the memory 9, executes an operating system and one or more applications 11. Some methods described elsewhere herein may be programmed as software instructions contained within the one or more applications 11 stored in the memory 9 and executable by the processor 8. In some embodiments, the one or more applications include health tracking applications (e.g., Argus, Apple Health Data, LifeTrak, New Balance LifeTRNr+, Jawbone, Runkeeper, Withings, etc.) on the computing device 4, for example to acquire datatypes for one or more tiles.

The processor 8 is configured to execute one or more sets of instructions to effect the functioning of the computing device 4. In some embodiments, a set of instructions effects rendering of a GUI 10, analyzing a datatype or a health-related parameter, adding data to the GUI 10, dynamically rearranging the GUI 10, displaying one or more GUI 10 views, calculating one or more datatypes or health-related parameters (e.g., weight, insulin dose, glucose level, caloric budget, calories consumed, etc.), or any other output. In some embodiments, the processor 8 includes bar code, optical character recognition (OCR), or image recognition capability, for example to determine a weight of a user using a picture of a scale, a calorie content of a food or beverage using a picture of a product label, and/or a blood pressure of a user using a picture of the blood pressure gauge.

The display 6 of the computing device 4 is configured to present one or more GUIs 10 and/ or receive one or more inputs from a user. In some embodiments, the display 6 includes a Thin Film Transistor liquid crystal display (LCD), in-place switching LCD, resistive touchscreen LCD, capacitive touchscreen LCD, organic light emitting diode (LED), Active-Matrix organic LED (AMOLED), Super AMOLED, Retina display, Haptic/Tactile touchscreen, and/or Gorilla Glass. The GUI 10 may include controls, which enable a user to interact with the GUI 10. The GUI 10 may include buttons, sliders, toggle buttons, toggle switches, switches, dropdown menus, combo boxes, text input fields, check boxes, radio buttons, picker controls, segmented controls, steppers, and/or any other type of control. In some embodiments, the user may use different tactile or haptic lengths or pressures to navigate on the display. For example, a user may use a short press, long press, light press, or forceful press to navigate on the display 6.

In some embodiments, the components 7 of the computing device 4 include an accelerometer, gyroscope, Global Positioning System (GPS), compass, barometer, camera, flash, speaker, microphone, barcode scanner, Quick Reference (QR) code reader, timer, pedometer, and/or any other type of sensor or chip. In some embodiments, the components 7 in the computing device are used to measure one or more datatypes.

For example, in some embodiments, an accelerometer measures changes in acceleration indicative of one or more steps of a user, other repetitive motions associated with physical activity, a sleep stage of a user (e.g., REM versus non-REM cycles), and/or a change in position of the user (e.g., standing versus sitting). In various embodiments, the computing device 4 is programmed to act as a pedometer and count a user's total number of steps based on signals received from the accelerometer. In some embodiments, the computing device 4 is programmed to track a duration of time the user is engaged in physical activity based on signals received from the accelerometer.

Further, for example, in some embodiments, a camera of the computing device, may be used to measure a heart rate of the user (e.g., using a sequence of images or video). In some such embodiments, the computing device receives an input including, for example, a sequence of images or video of the user's face or a fingertip or finger pad of the user. The sequence of images or video from the computing device is analyzed, for example by the processor, to determine a heart rate of the user. For example, in some embodiments, the processor detects and analyzes changes in facial coloration and/or light reflected to provide a heart rate reading. Alternatively, in some embodiments, changes in skin color, transparency, and/or light reflected caused by blood flow in the fingertip or finger pad are tracked and analyzed over time to determine a heart rate of the user. Alternatively or additionally, in some embodiments, a camera of the computing device is used to photograph one or more beverages or foods consumed by the user, for example to determine calories consumed; and/or photograph a readout of a scale, for example, to determine a weight of a user.

Further, for example, in some embodiments, one or more speakers and one or more microphones are used to determine a sleep duration and quality of the user, for example, using the Sonar Effect. In such embodiments, the computing device emits inaudible sound signals from one or more speakers and acquires sound signals through one or more microphones to measure the change in amplitude, time delay, and/or frequency relative to the source audio signal in order to determine locations and movements of the objects (e.g., a person while sleeping). The sound waves emitted by the one or more speakers bounce off the walls, any objects, and any people in the room, and are returned to and acquired by the one or more microphones. If there is movement in the room, for example, as a result of a person moving while sleeping in bed, it will cause a change in amplitude, time delay and/or frequency of the returned sound waves.

In some embodiments, a user's relative amount of movement is monitored throughout an entire period of time in which the sleep tracking functionality is active (e.g., from the time a user turns on the sleep tracking functionality when getting into bed until the user turns off the functionality when waking for the day). In such embodiments, the one or more speakers emit sound signals at a regular interval, for example, every five seconds, every ten seconds, every minute, every two minutes, or other time interval. In other embodiments, a user's relative amount of movement is only monitored via the Sonar. Effect upon activation by a trigger. In some such embodiments, the detection of a noise (e.g., caused by a movement of the user), the detection of a dark room, the absence of noise (e.g., in a quiet room for sleeping), or the setting of an alarm by the user serves as the trigger. In such embodiments, the one or more speakers may emit sound signals upon activation by the trigger. In some embodiments, the one or more speakers may emit sound signals at a regular interval for a set amount of time following a trigger. The microphone may be activated throughout the duration of the sleep tracking to “listen for” (i.e., acquire) sound signals. Alternatively, the microphone may only be activated with activation of the speakers, remaining active for a period of time following emission of a sound wave from the speakers.

In some embodiments, the computing device 4 is programmed to break down and categorize segments of time during the sleep tracking period based on the user's quality of sleep during those segments. For example, in some embodiments, the user's relative amounts of movement are used to segregate the sleep tracking period into segments having labels such as: awake, restless, light sleep, deep sleep, REM sleep, non-REM sleep, etc. In some embodiments, the computing device 4 is programmed to count the number of times a user awakens or is restless during the night; in some embodiments, the computing device 4 is programmed to total the amount of time the user was awake or restless and the amount of time the user experienced high-quality, deep sleep. In some embodiments, the computing device 4 is programmed to pair a time of night (e.g., 10 PM, 12 AM, etc.) with the quality of sleep of the user. In some embodiments, the range of sound frequencies emitted from the one or more speakers is 15 kHz to 50 kHz; in one embodiment, the sound frequency emitted from the one err more speakers is approximately 20 kHz.

Further, for example, a barcode scanner may be used to scan a barcode on one or more beverages or foods consumed by the user, for example to determine calories consumed. Further, for example, a compass or GPS may be used to determine an exercise route or a distance of an exercise. Further, for example, a user may “add a glass of water” to their water consumption tile using a gesture (e.g., tilting the phone), activating the accelerometer or gyroscope in the phone.

In some embodiments, a power supply, such as a battery 13 is included within the computing device 4 and is electrically coupled to provide power to the processor 8 and other electronic components. The battery 13 may be rechargeable or disposable.

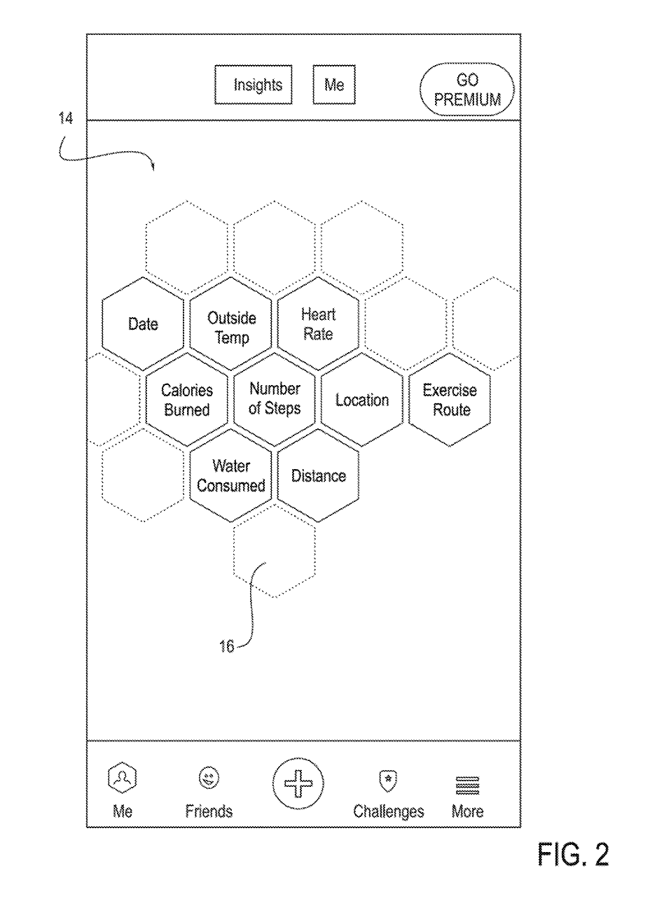

In some embodiments, as shown in FIGS. 2-5, a graphical user interface (GUI) for presentation on a mobile computing device includes a first plurality of hexagonal tiles 14 arranged in a honeycomb pattern, each hexagonal tile 16 representing a tracked data point. The first plurality of hexagonal tiles 14 together display tracked data for a plurality of datatypes. Further, the GUI is configured to dynamically rearrange the first plurality of hexagonal tiles in response to an input to maintain a sequence of tracked data. The GUI of FIGS. 2-5 functions to maximize the amount of data displayed on the GUI, so that a user does not need to scroll repeatedly or repeatedly select icons or links to navigate around in the application to vie review, use, and/or interact with the tracked data.

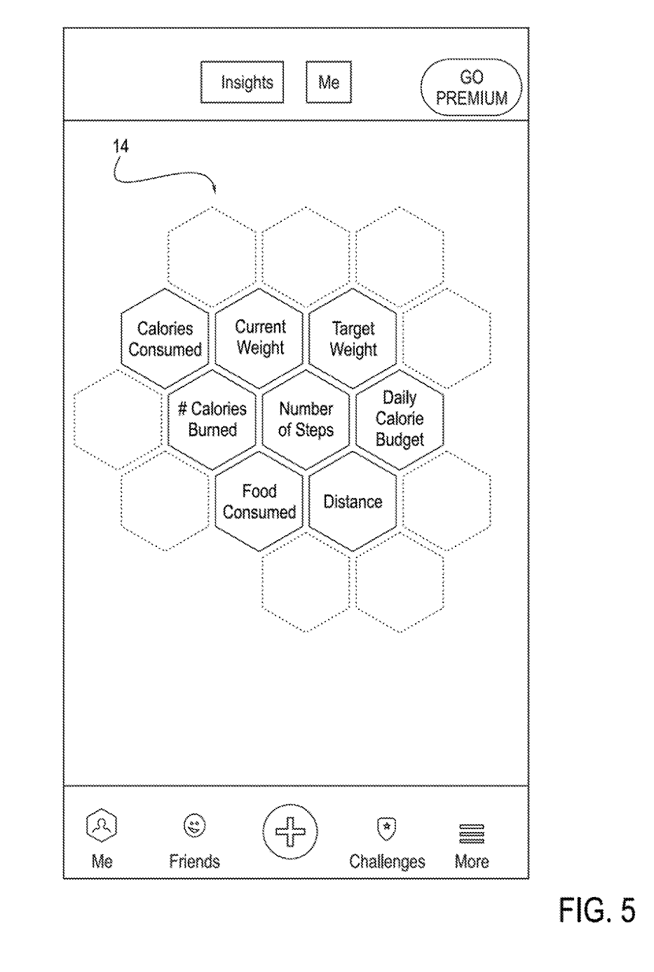

For example, as shown in FIGS. 2-5, each hexagonal tile 16 of the GUI represents a datatype or health-related parameter. In some embodiments, a user may select which hexagonal tiles are displayed or the system may automatically display a set of hexagonal tiles based on user preferences, a user profile, or data acquired. In some embodiments, the datatype includes diabetes tracking and/or management as shown in FIG. 3, heart health tracking and/or management as shown in FIG. 4, or weight tracking and/or management as shown in FIG. 5.

In some embodiments, the hexagonal tiles dynamically rearrange. The hexagonal tiles may dynamically rearrange in response to an input. An input may include data received by the system front a sensor, camera, etc.); data received from an external device; data received from an application; user interaction with the system, for example a user selecting a tile for the filtered view; and/or a system alert to alert the user to, for example, a threshold reached or exceeded by a tracked datatype represented in a tile, an achieved goal, or an unhealthy condition (e.g., heart rate, blood pressure, standup test result, etc.). In some embodiments, the hexagonal tiles dynamically rearrange to maintain a time-ordered sequence most recently updated to least recently updated), user-ordered sequence (e.g., indicated by user or based on a profile of a user), or a task driven sequence (e.g., reminders, to-do list, daily goals. etc.). As an example of time-ordered sequence rearrangement, after the user completes a run, the tiles may dynamically rearrange to reposition the heart rate, distance, step count, and route tiles at the top of the honeycomb configuration. As an example of user-ordered sequence rearrangement, a user may specify in her profile or through a series of tile selections that she desires to track her heart health. The tiles may dynamically rearrange to position the heart rate, pulse shape/waveform, and standup test tiles at the top of the honeycomb configuration. As an example of task driven sequence, a diabetic user may need reminders for insulin injections, meal timings, and calorie consumption monitoring. The tiles may dynamically arrange to position an insulin injection counter, a glucose level check reminder, meal timer, and calorie counter tiles at the top of the honeycomb configuration.

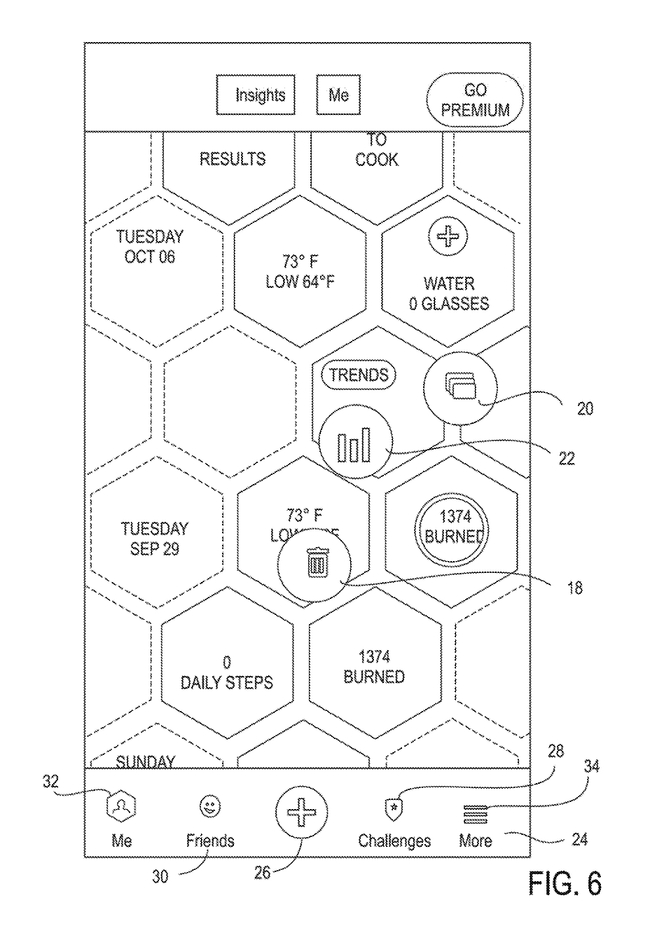

Further, each of the plurality of hexagonal tiles is configured to be activated by a user selection. As shown in FIG. 6, activating the tile functions to display one or more additional menu options, features, or icons to the user, for example to delete a tile 18, add a tile, present a filtered view 20, present a trend view 22, and/or to share a tile with fiends or a social network. In some embodiments, a tile is activated by a user by touch or gaze. For example, a user may activate a tile by performing a long press on the tile or a more forceful press on the tile, for example using Apple 3D touch or force touch technology. Further for example, a user may activate a tile by directing his/her gaze towards the tile of interest. Gaze direction detection may be determined by tracking eye movement with video or using search coils or electrooculogram. Each icon, option, or feature is configured to route a user to the corresponding view upon selection, or perform the requested task (e.g., delete, add, etc.) upon selection.

In some embodiments, as shown in FIG. 6, one or more options or features may be present in a menu 24. For example, one or more options or features may include add a tile 26, challenge a friend or contact to a challenge 28, add or find friends 30 (e.g., using contact lists, social networks, searches, hashtags, names, etc.), and/or create a profile 32. In some embodiments, a “more” link 34 allows a user to view his/her profile, goals, friends and followers, charts, trends, and/or any other user-related information. In some embodiments, a “more” link 34 allows a user to view or read one or more articles, tips, and/or tricks to achieving or maintaining health and/or fitness.

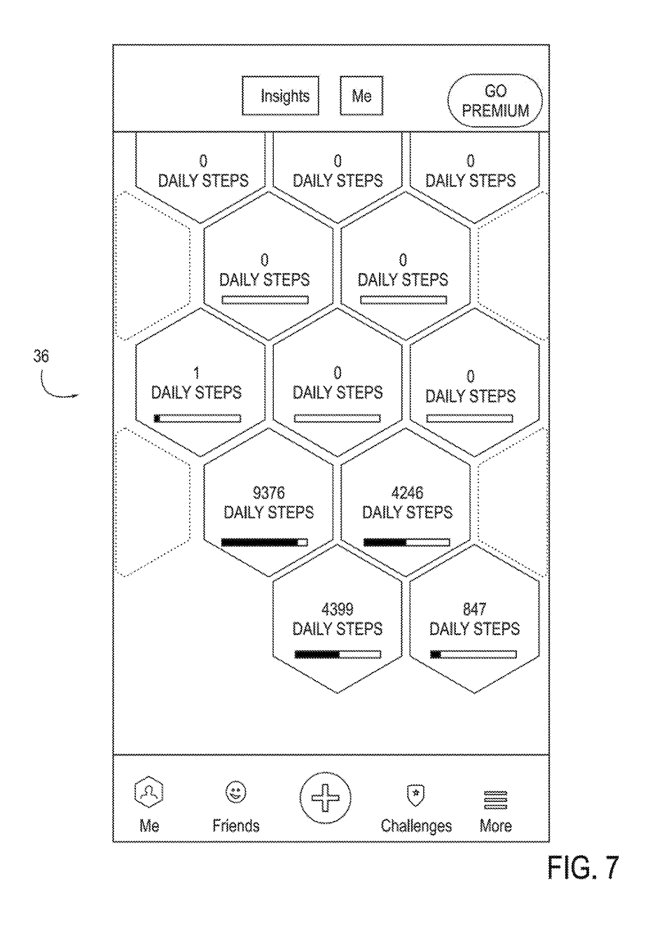

In some embodiments, as shown in FIG. 7, the filtered view 36 includes the honeycomb pattern with some of the first plurality of hexagonal tiles removed and rearranged so as to only display the user selected tile and other hexagonal tiles representing tracked data of a particular data type, for example related to the user selected tile. For example, if the user selects a step count tile, the system may display the user selected step count tile for the current day and several step count tiles for previous days, as shown in FIG. 7. As a further example, if the user selects a heart rate tile for inclusion in the filtered view, the system may also display heart rate variability and standup test result tiles in the filtered view as related content.

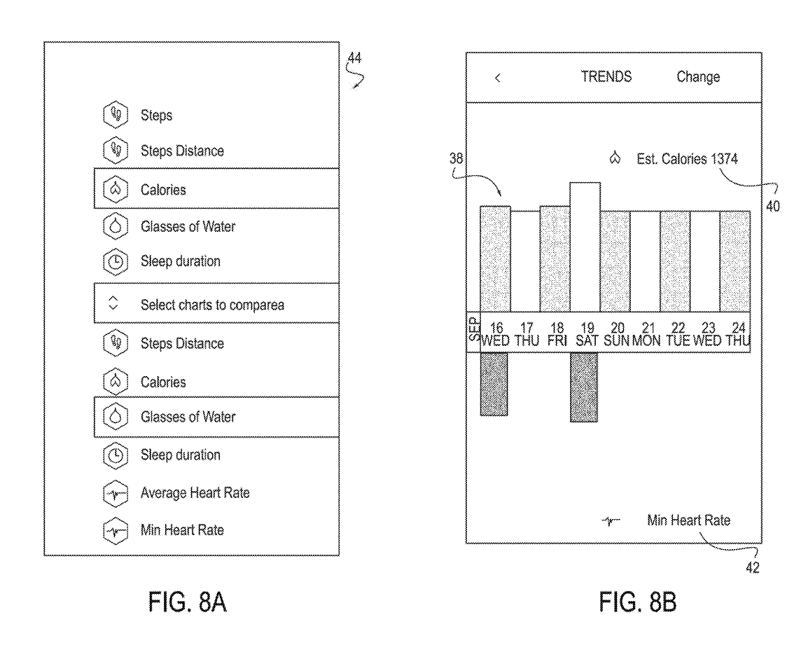

In some embodiments, as shown in FIGS. 8A and 8B, the trends view 38 includes a graph of tracked data of a first datatype 40 (e.g., user-selected or system-selected) plotted against time or against tracked data of a different datatype 42 specified by the user or the system. For example, a user may select to display his/her heart rate over time or to display his/her calories burned over steps counted using a comparison selection view 44, as shown in FIG. 8A. In some embodiments, when the user selects a tile for the trends view, the system may recommend one or more other tiles with which to compare the user selected tile in the trends view or the system may recommend plotting the user selected tile against time. In some embodiments, the data is displayed in a graph in a trends view as shown in FIG. 8B. For example, a first datatype is listed on the x-axis and a second datatype is listed on the y-axis. In some such embodiments, the selected data is displayed in histogram, as shown in FIG. 8B; a scatter plot; pie chart; pictograph; line graph; table; or any other format for displaying data comparatively.

In some embodiments, a user may desire to archive, delete, or otherwise remove from view one or more tiles. In some such embodiments, activating a tile may present a user with an option to delete, archive, or hide the tile from view.

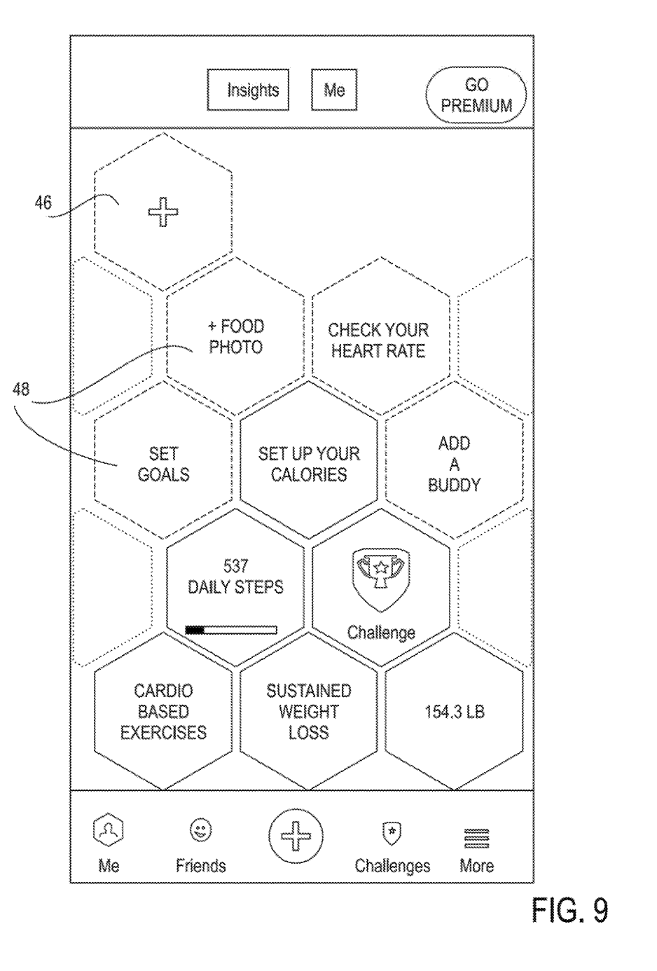

In some embodiments, as shown in FIG. 9, a user may desire to add a new tile 46 or reactivate an existing inactive tile for tracking data of a particular datatype. In some such embodiments, the tile may be recommended to the user by the system 48; the tile may be on a periphery of the honeycomb configuration in an inactive state (e.g., darkened, dull, paused, empty, shadowed, etc.); or the tile may appear, for example from a side of the display or from behind the honeycomb configuration.

In some embodiments, a user may desire to share the data tracked on a tile. In some such embodiments, the data on the tile may be configured to be uploaded to a social networking site, support group, a clinical server, or emailed or SMS messaged to a friend, family member, group, or clinician with or without additional commentary about the data. For example, a diabetes patient using the GUI described herein may desire to share one or more tiles or datatypes (e.g., glucose levels, injection amount, caloric intake, etc.) with a diabetes support group or a diabetes management or prediabetes prevention adviser. In some embodiments, sharing the data on a tile includes enabling commenting functionality, for example to enable other users of the social network or support group to provide commentary, feedback, positive reinforcement, or encouragement to the user.

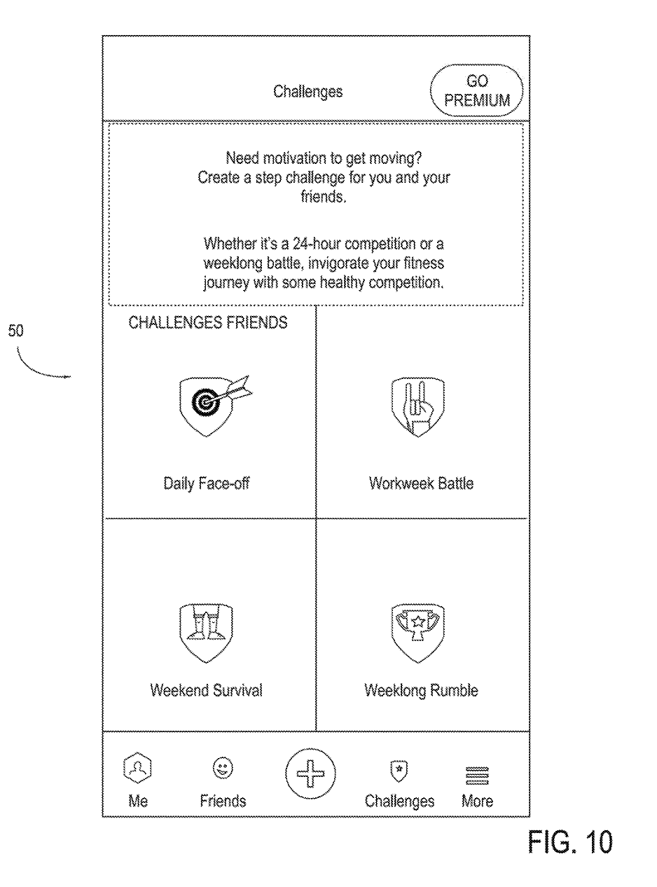

In some embodiments, as shown in FIG. 10, a competition view 50 may enable two or more users to share data on their respective tiles to compete over a period of time (e.g., day, week, weekend, month, etc.), for example to achieve a goal or a target. For example, two or more users may compete on a particular day or daily, during a weekend, over a five day period (e.g., work week), or over a seven day period (weeklong). For example, two or more users may compete to achieve the highest number of steps, most weight loss, most glasses of water, least amount of calories, etc.

In some embodiments, the GUI includes a second plurality of hexagonal tiles arranged in the honeycomb structure. The second plurality of hexagonal tiles represents recommended trackable datatypes. For example, if the user is not tracking water intake, weight, or calorie consumption, the system may actively (e.g., push notification, badge notification, etc.) recommend tracking these datatypes with one or more tiles or may passively provide the tiles on a periphery of the honeycomb configuration for activation by user selection or by an activity performed by the user and detected by the system, for example as shown in FIG. 9. For example, a step counter tile may not be activated until the user activates the tile by touch or by beginning to walk or run.

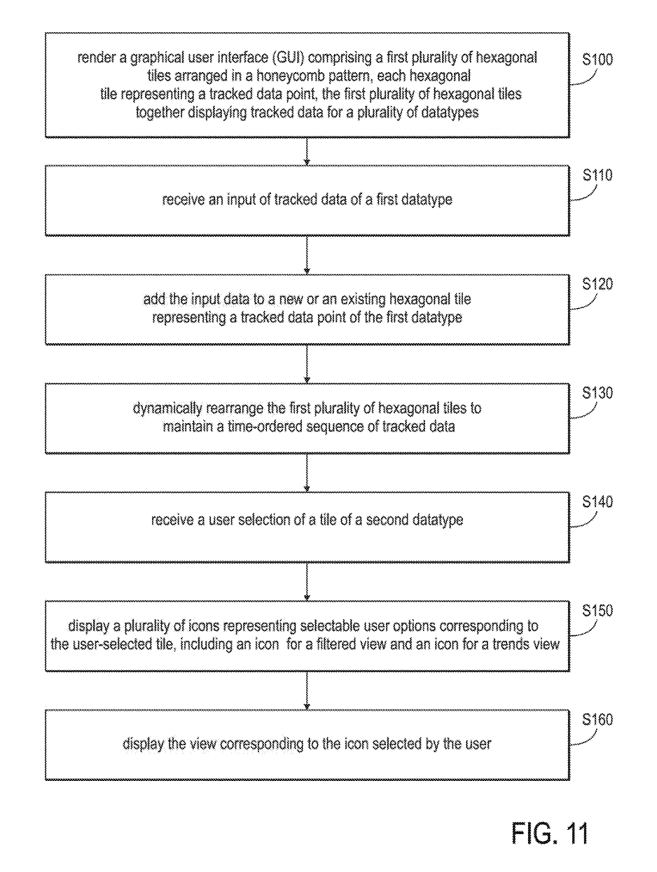

In some embodiments, as shown in FIG. 11, a method performed by a mobile computing device includes rendering a GUI, as shown in FIG. 2, including a first plurality of hexagonal tiles arranged in a honeycomb pattern, each hexagonal tile representing a tracked data point, the first plurality of hexagonal tiles together displaying tracked data for a plurality of datatypes S104; receiving an input of tracked data of a first datatype S110; adding the input data to a new or an existing hexagonal tile representing the tracked data point of a first datatype S120; dynamically rearranging the first plurality of hexagonal tiles to maintain a time-ordered sequence of tracked data S130; receiving a user selection of a tile of a second datatype S140; displaying a plurality of icons representing selectable user options corresponding to the user-selected tile, including an icon for a filtered view and an icon for a trends view S150; and displaying the view corresponding to the icon selected by the user S160. The method functions to display a plurality of hexagonal tiles and to update and or rearrange the tiles upon receiving an input from the user, the system, and/or an external device.

In some embodiments, the first datatype is the same as the second datatype. some such embodiments, for example, the tracked data of a first datatype may include heart rate and user selection of a tile of a second datatype may include a tile displaying heart rate. In some embodiments, the first datatype is different than the second datatype. In some such embodiments, for example, the tracked data of a first datatype may include heart rate and a user selection of a tile of a second datatype may include step count.

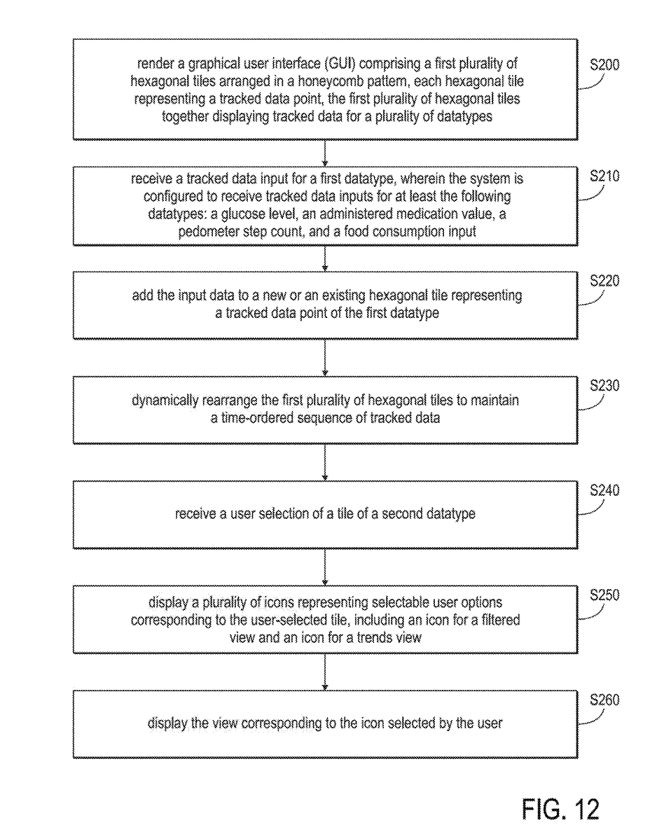

In some embodiments, as shown in FIG. 12, a method for managing diabetes includes rendering a GUI, as shown in FIG. 3, including a first plurality of hexagonal tiles arranged in a honeycomb pattern, each hexagonal tile representing a tracked data point, the first plurality of hexagonal tiles together displaying tracked data for a plurality of datatypes S200; receiving a tracked data input for a first datatype, such that the system is configured to receive tracked data inputs for at least the following datatypes: a glucose level, an administered medication value, a pedometer step count, and a food consumption input S210; adding the input data to a new or an existing hexagonal tile representing a tracked data point of the first datatype S220; dynamically rearranging the first plurality of hexagonal tiles to maintain a time-ordered sequence of tracked data S230; receiving a user selection of a tile of a second datatype S240; displaying a plurality of icons representing selectable user options corresponding to the user-selected tile, including an icon tar a filtered view and an icon for a trends view S250; and displaying the view corresponding to the icon selected by the user S260. The method functions to track, display, and/or rearrange one or more diabetes specific parameters.

As shown in FIG. 12, a method for managing diabetes includes S210, which recites receiving a tracked data input for a first datatype, such that the system is configured to receive tracked data inputs for at least the following datatypes: a glucose level, an administered medication value (e.g., insulin, glucagon, etc.), a pedometer step count, and a food consumption input. S210 functions to enable a diabetes patient, support group, and/or healthcare provider to monitor, track, or assess one or more diabetes specific parameters. In some embodiments, the method further tracks and/or calculates an ale level, an insulin to carbohydrate ratio, and/or a sleep cycle or quality of sleep of the user. In some embodiments, the method includes receiving one or more blood glucose levels from a blood glucose monitor, continuous glucose monitor, or interstitial glucose monitor communicatively coupled to the system. In some embodiments, the method includes receiving one or more administered medication values, for example from an insulin pen or other drug delivery device communicatively coupled to the system. In some embodiments, receiving a food consumption input includes receiving a photo of the food or product label, scanning a barcode of the food, or inputting a name of the food and determining the calorie and/or carbohydrate content of the food. In some such embodiments, the picture, barcode, or name of the food is deciphered using OCR and/or compared to a look-up table, menu options at local restaurants or diners, or search results, for example from the Internet or a database communicatively coupled to the system, to determine the calorie and/or carbohydrate content. In some embodiments, the method includes providing a mechanism (e.g., data entry field) for the user to enter the calorie and/or carbohydrate content into the system. In some embodiments, one or more tiles may automatically update upon receiving the tracked data: in some embodiments, manually inputting the tracked data is required to update the one or more tiles.

In some embodiments, the method includes sending tile updates to one or more healthcare professionals or support groups, for example to track the health of the diabetes patient. For example, a tile may be updated to show that patient's blood glucose level is above a safe threshold, and, thus, a notification may be sent to the patient's healthcare professional to alter the unsafe blood glucose level. In some embodiments, the method further includes providing a tile for communicating (e.g., via SMS, phone, email, Google Hangout, Facebook®, etc.) with a support group and/or healthcare professional.

In some embodiments, the method includes displaying a second plurality of hexagonal tiles arranged in the honeycomb structure of the GUI, the second plurality of hexagonal tiles representing recommended trackable datatypes recommended for the user by a diabetes management or prediabetes prevention adviser. For example, a diabetes management or prediabetes prevention adviser may request that the patient/user track his/her water intake and/or step count to gauge an activity level of the patient.

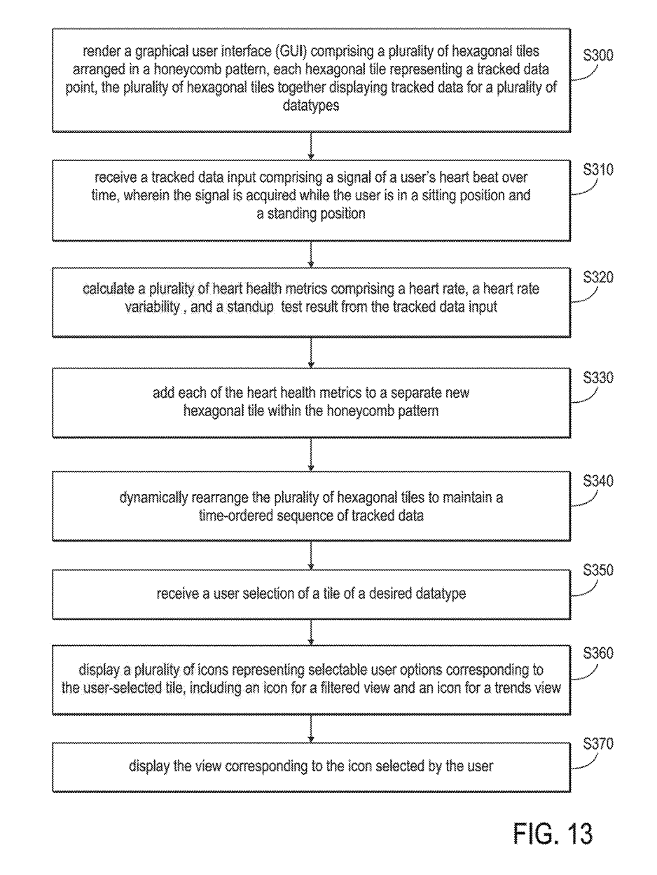

In some embodiments, as shown in FIG. 13, a method for heart health mobile tracking includes rendering a GUI, as shown in FIG. 4, comprising a plurality of hexagonal tiles arranged in a honeycomb pattern, each hexagonal tile representing a tracked data point, the plurality of hexagonal tiles together displaying tracked data for a plurality of datatypes S300; receiving a tracked data input comprising a signal of a user's heart heat over time, wherein the signal is acquired while the user is in a sitting position and a standing position S310; calculating a plurality of heart health metrics comprising a heart rate, a heart rate variability, and a standup test result from the tracked data input S320; adding each of the heart health metrics to a separate new hexagonal tile within the honeycomb pattern S330; dynamically rearranging the plurality of hexagonal tiles to maintain a time-ordered sequence of tracked data S340; receiving a user selection of a tile of a desired datatype S350; displaying a plurality of icons representing selectable user options corresponding to the user-selected tile, including an icon for a filtered view and an icon for a trends view S360; and displaying the view corresponding to the icon selected by the user S370. The method functions to track and display one or more heart health specific parameters.

As shown in FIG. 13, a method for heart health mobile tracking includes S310, which recites receiving a tracked data input comprising a signal of a user's heart beat over time, wherein the signal is acquired while the user is in a sitting position and a standing position; and S320, which recites calculating a plurality of heart health metrics including a heart rate, a heart rate variability, and a standup test result from the tracked data input. S310 and S320 function to acquire a user's heart rate to calculate, for example a heart rate variability and/or standup test results to determine a stress level of the heart. In some embodiments, heart rate or beat is determined using an external device, for example a FitBit or heart rate monitor. In some embodiments, heart rate or beat is determined using the pulse rate captured using a camera of the computing device. In some embodiments, the method includes determining a stress level of a user using the heart rate variability or standup test results as an input.

In some embodiments, calculating and/or determining a heart variability of the user includes determining an average heart rate variability of the user by comparing the time between adjacent heartbeats over a defined time period (e.g., hours, days, weeks, etc.). Once a baseline heart rate variability of the user is established, the method may include comparing the user's current heart rate variability to the user's average heart rate variability. In some embodiments, a user's baseline heart rate variability is unavailable. In some such embodiments, an average heart rate variability for the user's age group, ethnic group, weight group, socioeconomic group, or any other comparative group may be used. In some embodiments, the method includes providing an interpretation of the heart rate variability results. For example, low heart rate variability indicates higher heart stress while higher heart rate variability indicates lower heart stress.

In some embodiments, calculating and/or determining standup test results for a user includes determining a resting heart rate of the user, for example while the user is sitting or in a reclined position, and a heart rate of the user while the user transitions from the sitting or reclined position to standing. In some embodiments, the method includes determining a heart health of the user using standup test results and/or providing an interpretation of the standup test results. For example, a low resting heart rate and a strong heart rate spike in response to standing, indicates a healthier heart (i.e. low stress) and/or good hydration. In contrast, for example, a small heart rate spike indicates higher heart stress and/or poor hydration. In some embodiments, the method includes notifying a user of a stressed heart condition. For example, the system may notify the user that the user should seek hydration or the user should rest or relax. The notification may include an SMS, email, push notification, or a badge notification appearing on the tile. In some embodiments, the method includes recommending to the user that the user contact a healthcare professional regarding the user's stressed heart state. In some embodiments, the method includes providing contact information for one or more healthcare providers or professionals near the user.

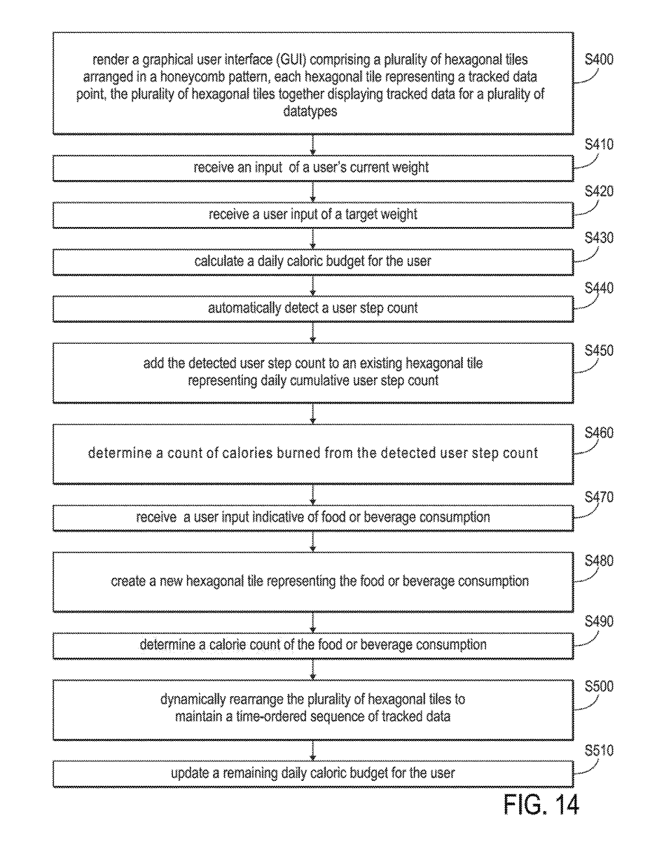

In some embodiments, as shown in FIG. 14, a method for weight management includes rendering a GUI, as shown in FIG. 5, comprising a plurality of hexagonal tiles arranged in a honeycomb pattern each hexagonal tile representing a tracked data point, the plurality of hexagonal tiles together displaying tracked data for a plurality of datatypes S400; receiving an input of a user's current weight S410; receiving a user input of a target weight S420; calculating a daily caloric budget for the user S430; automatically detecting a user step count S440, for example using a pedometer; adding the detected user step count to an existing hexagonal tile representing daily cumulative user step count S450; determining a count of calories burned from the detected user step count S460; receiving a user input indicative of food or beverage consumption S470; creating a new hexagonal tile representing the food or beverage consumption S480; determining a calorie count of the food or beverage consumption S490; dynamically rearranging the plurality of hexagonal tiles to maintain a time-ordered sequence of tracked data S500; and updating a remaining daily caloric budget for the user S510. The method functions to track and display one or more heart health specific parameters.

As shown in FIG. 14, a method for weight management includes S410, which recites receiving an input of a user's current weight; and S420, which recites receiving a user input of a target weight. S410 and S420 function to assess a user's current and target weight to help a user reach his/her weight goals. In some embodiments, receiving an input of a user's weight includes receiving a numerical value from a scale communicatively coupled to the computing device; receiving from a user's manual entry a numerical value indicative of the user's weight; or receiving from a remote server (e.g., from a clinic or other database) a numerical value indicative of a user's weight. In some embodiments, receiving a user input of a target weight includes receiving from a user's manual entry a numerical value indicative of the user's desired or target weight; or receiving from a remote server (e.g., from a clinic, health provider, etc.) a numerical value indicative of the user's desired or target weight. In some embodiments, the method includes receiving an input for height and weight, for example from the user, a clinic, or an external device; determining a target weight for the user; and recommending a target weight to the user.

As shown in FIG. 14, a method for weight management includes S430, which recites calculating a daily caloric budget for the user. S430 functions to help a user to determine and stay within a specific calorie budget tailored to the user. In some embodiments, the method includes calculating, using the processor, a number of calories required per day, for example by multiplying the target bodyweight of the user by ten if the user is a female or eleven if the user is a male. In some embodiments, the method includes calculating, using the processor, a daily caloric budget for the user using a basal metabolic rate calculator, the Sterling-Pasmore equation, and/or the Harris Benedict formula.

As shown in FIG. 14, a method for weight management includes S460, which recites determining a count of calories burned from the detected user step count. In some embodiments, determining a count of calories burned from the detected user step count includes multiplying, using the processor, the user's step count by an average number of calories burned per step, for example from a database, based on past user data, and/or using relevant group data (e.g., age, ethnicity, socioeconomic, etc.). In some embodiments, the method includes determining a user specific calories burned per step calibration feature. For example, the system, using the processor, may multiply a user's weight by a scaling factor (e.g., 0.57 based on 2 mph walking pace) to determine the number of calories the user burns per mile; record the number of steps the user takes in one mile using, for example a pedometer and GPS; and divide the number of calories the user burned in one mile by the number of steps the user takes in one mile. In some embodiments, the method uses an approximation to determine an amount of calories burned. For example, 10,000 steps equal approximately five miles, which equals approximately 500 calories burned.

As shown in FIG. 14, a method for weight management includes S470, which recites receiving a user input indicative of food or beverage consumption; and S490, which recites determining a calorie count of the food or beverage consumption. S470 and S490 function to help a user track daily caloric intake. In some embodiments, the method includes receiving one or more photos of beverage(s) or food(s) the user is about to consume; and determining and/or calculating a calorie content of the beverage(s) and/or food(s) based on the one or more photos. In some embodiments, the method includes receiving one or more descriptions of beverage(s) or food(s) the user is about to consume; and determining and/or calculating a calorie content of the beverage(s) and/or food(s) based on the one or more descriptions, for example using a database or a look-up table. In some embodiments, the method includes scanning one or more barcodes or QR codes of beverage(s) or food(s) the user is about to consume; and comparing the scanned data about the beverage(s) or food(s) to one or more calorie content databases to determine a calorie content of the beverage(s) and/or food(s). In some embodiments, the method includes receiving one or more photos of a product label of beverage(s) and/or food(s) a user is about to consume; and deciphering, using for example OCR, a calorie content of the beverage(s) and/or food(s) based on the product label.

As shown in FIG. 14, a method for weight management includes S510, which recites updating a remaining daily caloric budget for the user. S510 functions to notify the user of the amount of calories the user can consume during the remainder of the day to stay within his/her calorie budget for the day. In some embodiments, the method includes determining a difference between daily calorie budget calculated in S430 and the consumed calorie budget in S470 and S490. In some embodiments, the method includes subtracting burned calories in S460 from the consumed calorie budget in S470 and S490.

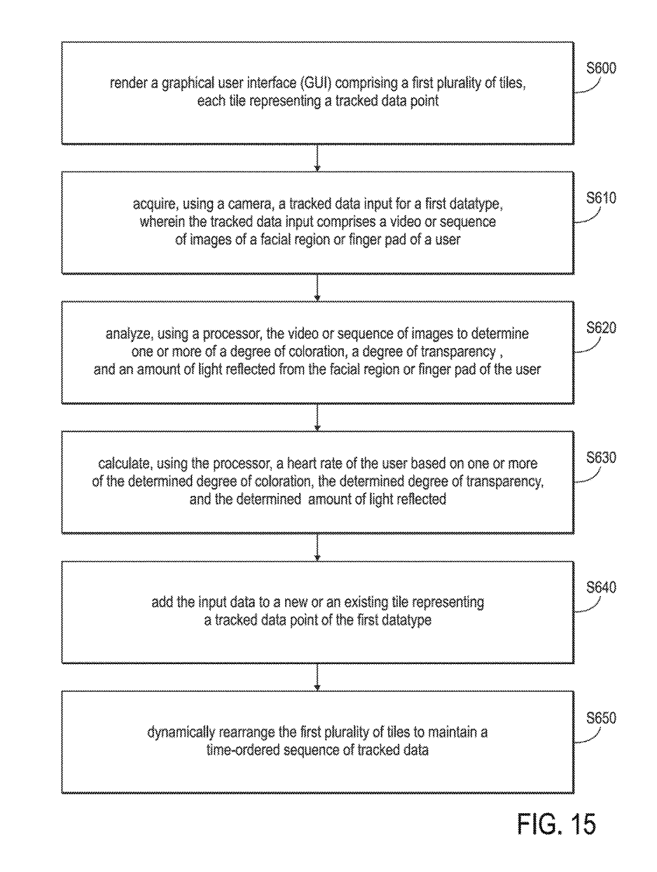

In some embodiments, as shown in FIG. 15, a method for heart rate monitoring includes rendering a graphical user interface (GUI) comprising a first plurality of tiles, each tile representing a tracked data point S600; acquiring, using a camera, a tracked data input for a first datatype, wherein the tracked data input comprises a video or sequence of images of a facial region or finger pad of a user S610; analyzing, using a processor, the video or sequence of images to determine one or more of a degree of coloration, a degree of transparency, and an amount of light reflected from the facial region or finger pad of the user S620; calculating, using the processor, a heart rate of the user based on one or more of the determined degree of coloration, the determined degree of transparency, and the determined amount of light reflected S630; and adding the input data to a new or an existing tile representing a tracked data point of the first datatype S640. In some embodiments, each tile is a hexagonal tile, and the plurality of tiles is arranged in a honeycomb configuration, as described elsewhere herein; in some embodiments, adding the input data to a new or existing tile at S640 causes the plurality of tiles to be dynamically rearranged to maintain a time-ordered sequence of tracked data S650.

As shown in FIG. 15, a method for heart rate monitoring includes S620 and S630, which recite analyzing, using a processor, the video or sequence of images to determine one or more of a degree of coloration, a degree of transparency, and an amount of light reflected from the facial region or finger pad of the user; and calculating, using the processor, a heart rate of the user based on one or more of the determined degree of coloration, the determine degree of transparency, and the determined amount of light reflected. In some embodiments, S620 and S630 include determining an amount of light reflected from the facial region of the user using the video or sequence of images, wherein increased blood volume expands blood vessels in the facial region with every heart beat causing more light to be absorbed, resulting in a decrease in the amount of light reflected from the facial region. In some embodiments, S620 and S630 include detecting one or more changes in skin color and transparency caused by blood flowing in the fingertip or finger pad of the user.

In some embodiments, the method of FIG. 15 includes illuminating a facial region or finger pad of a user, acquiring a video or series of images, and measuring one or more changes in light absorption across the video or series of images. In some embodiments, the method of FIG. 15 includes filtering one or more signals from the acquired and analyzed video or series of images to remove artifacts due to face or finger movement, changes in venous pressure, and/or other high frequency noise.

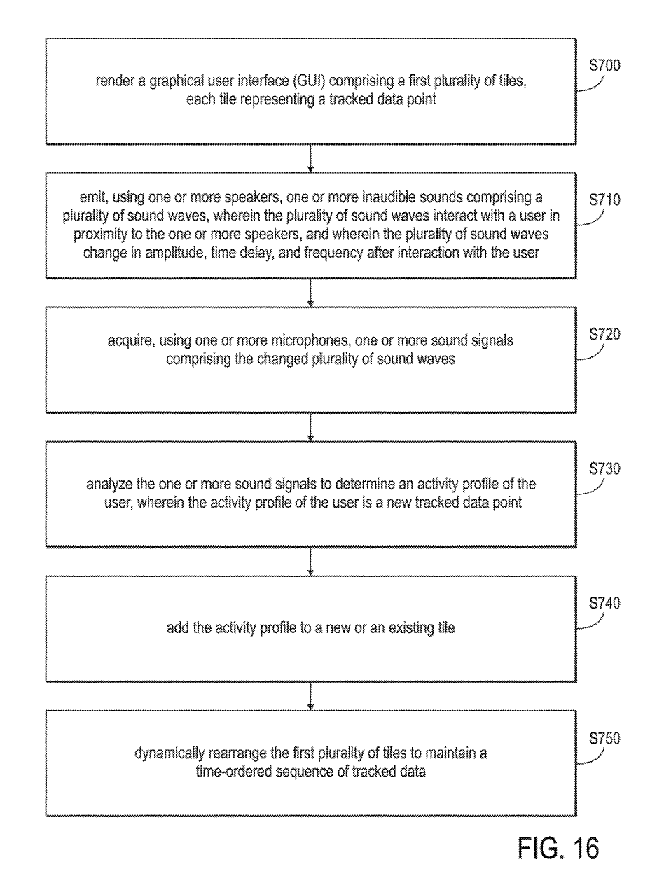

In some embodiments, as shown in FIG. 16, a method of sleep quality monitoring performed by a computing device includes rendering a graphical user interface (GUI) comprising a first plurality of tiles, each tile representing a tracked data point S700; emitting, using one or more speakers, one or more inaudible sounds comprising a plurality of sound waves, wherein the plurality of sound waves interact with a user in proximity to the one or more speakers, and wherein the plurality of sound waves change in amplitude, time delay, and/or frequency after interaction with the user S710; acquiring, using one or more microphones, one or more sound signals comprising the changed plurality of sound waves S720; analyzing the one or more sound signals to determine an activity profile of the user, wherein the activity profile of the user is a new tracked data point S730; and adding the activity profile to anew or an existing tile S740. In some embodiments, each tile is a hexagonal tile, and the plurality of tiles is arranged in a honeycomb configuration, as described elsewhere herein; in some embodiments, adding the activity profile to a new or existing tile at S740 causes the plurality of tiles to be dynamically rearranged to maintain a time-ordered sequence of tracked data S750.

As shown in FIG. 16, a method of sleep quality monitoring includes functional block 5710, which recites emitting, using one or more speakers, one or more inaudible sounds comprising a plurality of sound waves. The plurality of sound waves interacts with a user in proximity to the one or more speakers and experiences a change in amplitude, time delay, and/or frequency from the interaction. At functional block S720, the computing device acquires, using one or more microphones, one or more sound signals comprising the changed plurality of sound waves. In some embodiments, the plurality of sound waves emitted by the one or more speakers of the computing device bounce off of one or more walls, objects, or users in a room, area, or location. As a result of interaction with or bouncing off of the one or more walls, objects, or users in the room, the plurality of sound waves change, alter, or shift in amplitude, time delay, and/or frequency. This change in amplitude, time delay, and/or frequency is analyzed in the acquired sound signals to determine an activity profile of the user, as shown in S730. For example, the computing device is programmed to register no activity for a user if the change in amplitude, time delay, and/or frequency remains constant, and the computing device is programmed to detect movement of a user if there is a change, over time, of the detected amplitude, time delay, and/or frequency. In some embodiments, the activity profile includes a sleep pattern of the user, for example, including awake periods or restless periods, or periods of light sleep or deep sleep, or periods of REM sleep or non-REM sleep. In some embodiments, the method includes displaying the activity profile of the user as a function of time, for example to determine a timeframe in which the user experiences, for example, deep sleep or light sleep.

In some embodiments, the method of FIG. 16 includes tracking an activity profile of a user upon activation by a user input, a scheduled bedtime of the user (e.g., 10 PM), or detection of an environmental condition such as a quiet room (e.g., reduced sound acquired by microphones in computing device) or dark room (e.g., reduced light acquired by camera in computing device). In some embodiments, the method includes ending tracking of an activity profile of the user upon activation of an alarm (e.g., to wake the user) on the computing device. In some embodiments, motion detection via the Sonar Effect is ongoing throughout a sleep tracking period, and in other embodiments, motion detection via the Sonar Effect is intermittent, for example, upon activation by a trigger, as described elsewhere herein. In some embodiments, the method includes waking a user from sleep, for example using an alarm or vibration, upon detecting a unique sleep pattern of the user in the user's activity profile, for example lighter sleep or non-REM sleep.

The systems and methods of the preferred embodiment and variations thereof can be embodied and/or implemented at least in part as a machine configured to receive a computer-readable medium storing computer-readable instructions. The instructions are preferably executed by computer-executable components preferably integrated with the system and one or more portions of the processor. The computer-readable medium can be stored on any suitable computer-readable media such as RAM, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

As used in the description and claims, the singular form “a”, “an” and “the” include both singular and plural references-unless the context clearly dictates otherwise. For example, the term “graphical user interface” may include, and is contemplated to include, a plurality of graphical user interfaces. At times, the claims and disclosure may include terms such as “a plurality,” “one or more,” or “at least one,” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed invention. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A diabetes management and prediabetes prevention system comprising:
a display;
a processor communicatively coupled to the display; and
a computer-readable medium having non-transitory, processor-executable instructions stored thereon, wherein execution of the instructions causes the processor to perform a method comprising:
rendering a graphical user interface (GUI) comprising a first plurality hexagonal tiles arranged in a honeycomb pattern, each hexagonal tile representing a tracked data point, the first plurality of hexagonal tiles together displaying tracked data for a plurality of datatypes;
receiving a tracked data input for a first datatype, wherein the system is configured to receive tracked data inputs for at least the following datatypes: a glucose level, an administered medication value, a pedometer step count, and a food consumption input;
adding the input data to a new car or an existing hexagonal tile representing a tracked data point of the first datatype;
dynamically rearranging the first plurality of hexagonal tiles to maintain a time-ordered sequence of tracked data;
receiving a user selection of a tile of a second datatype;
displaying a plurality of icons representing selectable user options corresponding to the user-selected tile, including an icon for a filtered view and an icon for a trends view; and
displaying the view corresponding to the icon selected by the user, wherein:
the filtered view comprises the honeycomb pattern with some of the first plurality of hexagonal tiles removed and rearranged so as to only display the user-selected tile and other hexagonal tiles representing tracked data of the second datatype, and
the trends view comprises a graph of tracked data of the second datatype plotted against time or against tracked data of a different datatype specified by the user.

2. The system of claim 1, wherein some or all of the tracked data is shareable with a diabetes management adviser or prediabetes prevention adviser.

3. The system of claim 2, wherein the system is configured to transmit and display messages sent between the diabetes management adviser or prediabetes prevention adviser and the user.

4. The system of claim 1, further comprising additional non-transitory, processor-executable instructions stored on the computer-readable medium, wherein execution of the additional instructions causes the processor to perform a method comprising: displaying a second plurality of hexagonal tiles arranged in the honeycomb structure of the GUI, the second plurality of hexagonal tiles representing recommended trackable datatypes, wherein each of the second plurality of hexagonal tiles is selectable to input and display data of the datatype represented by the selected tile.

5. The system of claim 4, wherein the second plurality of hexagonal tiles, represent trackable datatypes recommended for the user by a diabetes management adviser or prediabetes prevention adviser.

6. The system of claim 1, wherein the first datatype is the second datatype.

7. The system of claim 1, wherein the first and second datatypes are different datatypes.

8. The system of claim 1, wherein the system further comprises a blood glucose monitor communicatively coupled to the processor.

9. The system of claim 1, wherein the input is a user input.

10. The system of claim 1, wherein the input is data imported from a wirelessly connected device.

11. The system of claim 1, wherein the input is data acquired from a component of the mobile computing device.

12. The system of claim 1, wherein the display, processor, and computer-readable medium comprise components of a mobile computing device.

13. The system of claim 1 wherein the mobile computing device comprises a laptop, mobile phone, or tablet.

14. A method of diabetes management and prediabetes prevention comprising:
rendering a graphical user interface (GUI) comprising a first plurality of hexagonal tiles arranged in a honeycomb pattern, each hexagonal tile representing a tracked data point, the first plurality of hexagonal tiles together displaying tracked data for a plurality of datatypes;
receiving a tracked data input for a first datatype, wherein the system is configured to receive tracked data inputs for at least the following datatypes: a glucose level, an administered medication value, a pedometer step count, and a food consumption input;
adding the input data to a new or an existing hexagonal tile representing a tracked data point of the first datatype;
dynamically rearranging the first plurality of hexagonal tiles to maintain a time-ordered sequence of tracked data;
receiving a user selection of a tile of a second datatype;
displaying a plurality of icons representing selectable user options corresponding to the user-selected tile, including an icon for a filtered view and an icon for a trends view; and
displaying tine view corresponding to the icon selected by the user, wherein:
the filtered view comprises the honeycomb pattern with some of the first plurality of hexagonal tiles removed and rearranged so as to only display the user-selected tile and other hexagonal tiles representing tracked data of the second datatype, and
the trends view comprises a graph of tracked data of the second datatype plotted against time or against tracked data of a different datatype specified by the user.

15. The method of claim 14, further comprising transmitting one or more tile updates to a diabetes management adviser or prediabetes prevention adviser.

16. The method of claim 14, further comprising transmitting and displaying messages sent between the diabetes management adviser or prediabetes prevention adviser and the user.

17. The method of claim 14, further comprising displaying a second plurality of hexagonal tiles arranged in the honeycomb structure of the GUI, the second plurality of hexagonal tiles representing recommended trackable datatypes, wherein each of the second plurality of hexagonal tiles is selectable to input and display data of the datatype represented by the selected tile.

18. The method of claim 17, wherein the second plurality of hexagonal tiles represent trackable datatypes recommended for the user by a diabetes management adviser or prediabetes prevention adviser.

19. The method of claim 14, further comprising receiving the tracked data input from a glucose monitor or insulin delivery device.

20. The method of claim 14, further comprising receiving a recommendation for a trackable datatype from a diabetes management adviser or prediabetes prevention adviser.
